(19) United States
(12) Patent Application Publication
(10) Pub. No.: US 2002/0187119 A1
(43) Pub. Date: Dec. 12, 2002

(54) Deodorizing agent for restrooms

(76) Inventors: William R. Greer, Mauldin, SC (US); Edward M. Jamison JR., Simpsonville, SC (US)

Correspondence Address: Richard C. Litman, Litman Law Offices, Ltd., P.O. Box 15035, Crystal City Station, Arlington, VA, 22215-0035, US

(21) Appl. No.: 09/852,291

(22) Filed: May 10, 2001

(51) Int. Cl. A61L 9/015

(52) U.S. Cl. 424/76.2

(57) ABSTRACT

A single-use toilet deodorizing agent in the form of a solid tablet. The invention is a composition in tablet form made of citric acid, sodium bicarbonate, binding agent, dyes (optional), and a fragrance capable of being released upon dropping the composition in the water. The purpose of the tablet is to be portable and to mask unpleasant odors in the bathroom. The tablet can be deposited or delivered into the toilet water before, during, or after each use.

### CROSS-REFERENCE TO RELATED APPLICATION

This application claims the benefit of U.S. Provisional Patent Application Serial No. (unassigned), filed May 10, 2000.

### BACKGROUND OF THE INVENTION

1. Field of the Invention

The present invention relates generally to a deodorizing agent composition, and more specifically, a deodorizing agent composition in the form of a solid tablet for portability and for single-use in toilets. 2. DESCRIPTION OF RELATED ART

Deodorizing agents have been the subject of patents since the invention of the flush toilet. There are several problems associated with most deodorizing agents. Fixed dispensing apparatuses are usually required for dispersing deodorizing agents into the air of a bathroom. This is both expensive and labor intensive. Some deodorizing agents leave a permanent and everlasting odor which can be very undesirable.

The following patents describe the various types of deodorizing agents and similar chemical compositions.

U.S. Pat. No. 1,450,865, issued to Joseph Pelc on Apr. 3, 1923, describes a water-soluble product and process of making effervescent and non-effervescent tablets, lozenges, pills, candies, confections, powders, and similar products. The vehicle with which the active ingredients are mixed consists of an intimate mixture of any normal or hydrogen containing carbonate.

U.S. Pat. No. 3,242,093, issued to Walter A. Compton on Mar. 22, 1966, describes a composition for removing surface oxidation from metal surfaces. The patent uses a water-soluble composition which is useful for removing rust and corrosion from metallic objects and protects the cleaned surface from subsequent rusting and corrosion. The formulation comprises a four-gram tablet consisting of diammonium citrate, sodium bicarbonate, citric acid, and a surfactant.

U.S. Pat. No. 3,607,759, issued to Jordan B. Barth on Sep. 21, 1971, describes a denture soak tablet and the process of preparing a water-soluble effervescent tablet. The water-soluble ingredients include a carbonate, an organic acid and at least one oxidizing agent, and the terminal addition and blending of a water-soluble detergent.

U.S. Pat. No. 3,639,568, issued to William H. Schmitt on Feb. 1, 1972, describes gas-releasable and foamable compositions which are adapted to be packaged in conventional non-pressurized containers. The invention includes substantially anhydrous compositions containing a compressible water-insoluble gas in the form of an aliphatic hydrocarbon or halogenated hydrocarbon.

U.S. Pat. No. 4,180,467, issued to Jordan B. Barth on Dec. 25, 1979, describes a stable denture soak product which includes the properties of effervescence. The denture soak product consists of an acid anhydride, a solid organic acid, a solid inorganic carbonate, and oxidizing agents. The acid anhydride reduces caking tendencies by acting as an internal desiccant and increases the effervescence time of carbon dioxide evolution.

U.S. Pat. No. 4,567,613, issued on Feb. 4, 1986, and U.S. Pat. No. 4,633,533, issued on Jan. 6, 1987, to Frank Meeham describes a method and article for neutralizing offensive odors. The disposable article for neutralizing offensive odors comprises a sheet of foldable material. A portion of the sheet has a grid region for adsorption of an odor-neutralizing liquid.

U.S. Pat. No. 5,306,439, issued on Apr. 26, 1994, to Ronald R. Lockhart, describes a jewelry cleaner formulation with a molded wafer of citric acid and sodium bicarbonate compressed together to produce effervescence.

U.S. Pat. No. 5,307,525, issued to John W. O'Brien on May 3, 1994, describes a dispenser that injects a film into the toilet water to eliminate bathroom odors. The film includes a few drops of an appropriate fluid mixture of a chlorinated hydrocarbon containing a small amount of a volatile fragrant fluid which is added to the water in a toilet bowl prior to its use. The property of the fluid causes the resultant mixture to spread quickly over the entire surface of the water forming a non-permeable film through which the odor emanating from unflushed feces cannot pass.

U.S. Pat. No. 5,759,574, issued to Steven T. Bothe on Jun. 2, 1998, describes tablets which are placed in a toilet tank. The tablets are constructed of solid, clear or white cleansing and\or deodorizing chemicals and has a smaller color core in its center. Over time, the tablet dissolves to the center portion where a color is released indicating that another tablet needs to be added to the toilet tank.

U.S. Pat. No. 5,763,376, issued to Kenneth Ward et al. on Jun. 9, 1998, describes a lavatory cleaning block that releases a halogen-containing sanitizing agent in a controlled, substantially constant rate.

Great Britain Pat. Application No. 896,159, published on May 9, 1962, describes an improvement in or relating to descaling compositions. The compositions are for removing scale which is the incrustation of insoluble salts that is formed by evaporation of water.

France Pat. No. 1,127,352, published on Dec. 14, 1956, and an addition to the patent published on Jan. 9, 1959, describes a dispenser apparatus for deodorizing a bathroom.

An ideal deodorizing agent should be manufactured to be safe, portable, inexpensive, convenient, and efficient in temporarily deodorizing toilets.

None of the above inventions and patents, taken either singly or in combination, is seen to describe the instant invention as claimed.

### SUMMARY OF THE INVENTION

The single-use toilet deodorizing agent is a composition of citric acid, sodium bicarbonate, a binding agent such as corn syrup and corn starch, and a fragrance. A dye can be optionally included as well for aesthetic value.

The preferred shape of the pellet is a sphere or a disk. The tablet can be deposited or delivered into the toilet water either before, during, or after each use of the toilet fixture.

Accordingly, it is a principal object of the invention to provide a deodorizing agent that masks unpleasant odors in restrooms.

It is another object of the invention to provide a portable deodorizing agent in the form of a tablet which can be deposited or delivered into the water before, during, and after each use.

It is a further object of the invention to provide a deodorizing agent that can be easily carried on one's person or in a purse.

Still another object of the invention is to provide a deodorizing agent which is non-toxic and biodegradable in order to be safely flushed in a toilet.

It is an object of the invention to provide improved elements and arrangements thereof a deodorizing agent in an apparatus for the purposes described which is inexpensive, dependable and fully effective in accomplishing its intended purposes.

These and other objects of the present invention will become readily apparent upon further review of the following specification.

### DETAILED DESCRIPTION OF THE PREFERRED EMBODIMENTS

The present invention is a single-use toilet deodorizing agent in the form of a solid tablet. The invention is a chemical composition, in tablet form, including at least one dry organic acid, sodium bicarbonate, a binding agent, a colorful dye (optional), and a fragrance capable of being released upon dropping the composition in the water. The purpose of the tablet is to mask unpleasant odors in the bathroom. The tablet can be deposited or delivered into the toilet water either before, during, or after each use.

The tablet is designed to be easily carried on one's person or in a purse. However, use of the tablet after flushing is typically more effective. Upon dissolution, a fragrance will be evolved which disguises any toilet odor present. This can prevent uncomfortable or embarrassing situations.

The composition necessary for effervescence includes an acid and a base which react to form a gaseous product when introduced to water. Citric acid is a triprotic acid with a relatively low pKa of 3.1, and provides more protons per molecule for the reaction. This pKa value is lower than other commonly used organic acids such as carbonic acid, acetic acid, ascorbic acid or benzoic acid. Therefore, citric acid is the preferred acid, because less acid is required to neutralize the base for a given amount of sodium bicarbonate.

Sodium bicarbonate is relatively inexpensive, safe, and readily available. Sodium bicarbonate is very effective in absorbing unpleasant odors such as those emanating from a toilet after use. A stoichiometric calculation indicates that for every gram of citric acid that is used, 1.3 grams of sodium bicarbonate are required to completely neutralize the citric acid. If the citric acid remains in water as the monohydrate, then for every gram of citric acid used, 1.2 grams of sodium bicarbonate would be required. The effervescence quality of the tablet is primarily for aesthetic purposes, although this effervescence enhances the evolution of the fragrance and can speed the dissolution of the tablet.

Although the mixture can consist of any normal or hydrogen containing carbonate and any solid salt of any acid containing hydrogen, the preferred acid salt would be a citric acid salt. The effervescing salts are soluble in water and will form a product permanently stable in a dry atmosphere.

The preferred binding agents for binding the acid, base, dyes (optional), and fragrance are corn starch and corn syrup. Although any binding agent commonly known in the art can be utilized, corn starch and corn syrup are the preferred thickening and adhesive agents. If corn syrup is added, 20-45 wt. % of the total composition is suitable, but 35 wt. % is preferred. This chemical composition gives the pellet a texture which is firm, yet malleable, so that the pellet can be shaped into tablet form.

Other adhesives or binding agents that could be substituted for the corn starch or used in addition to these items include gelatin, gum arabic, pectin, cellulose, methylcellulose, ethylcellulose, sodium carboxymethylcellulose, carrageenan, amylose, sodium alginate, acacia, liquid glucose, and polyethylene glycols.

The preferred fragrance included in a tablet are ones used in food products, and can be introduced in solid or liquid form. Each fragrance is heavily scented and is intended to temporally mask any unpleasant odors. Some odors can be incidentally absorbed, which enhances the nature of the present invention. Preferably, one fragrance is included for any given tablet to maintain simplicity and lower costs, but a combination of fragrances can be used for aesthetic purposes.

The preferred fragrances included in the composition is selected from, but not limited to the following fragrances listed in Table 1.

TABLE 1

| COMMON NAME | CHEMICAL NAME |
|---|---|
| Cinnamon | Cinnamaldehyde |
| Vanilla | Vanillin |
| Peppermint | Menthol/Menthone |
| Wintergreen | Methyl salicylate |
| Banana | Isopentyl acetate |
| Pineapple | Ethyl butyrate |
| Orange | Octyl acetate |
| Apple | Methyl butyrate |
| Apricot | Pentyl propionate |
| Raspberry | Isobutyl formate |
| Rum | Ethyl formate |

A standard formulation for the deodorizing tablet is listed in Table 2. Each ingredient is listed along with their preferred relative amounts. The quantities can vary, depending on the size and shape of each tablet or the intensity of the fragrance desired.

TABLE 2

| INGREDIENTS | AMOUNT |
|---|---|
| Citric acid | 2 grams |
| Sodium bicarbonate | 2.4 grams |
| Fragrance(s) | 2 grams or 2 ml. |
| | (Each fragrance or |
| | combination) |
| Corn syrup | 5 grams |
| Corn starch | 7 grams |
| Dye (optional) | 2 drops |

For a colored pellet or a colored end product (in water), an appropriate food coloring dye can be added to the composition. Preferred dyes are included, but not limited to the dyes listed in Table 3. Otherwise, the pellet exhibits a pale yellow color. To produce the green color, equal amounts of yellow and blue dyes are combined.

TABLE 3

| DYE | COLOR PRODUCED |
|---|---|
| FD&C Red #40 | Red |
| FD&C Yellow #5 | Yellow |
| FD&C Blue #1 | Blue |
| FD&C Yellow #6 & Blue #1 | Green |

The manufacturing process includes the combining of an acid, a base, and a fragrance as dry ingredients, and thoroughly mixing such ingredients. Corn syrup is then added to form a cohesive substrate. At this point, the material is bound together, but remains extremely sticky. Corn starch is then added which thickens the mixture and provides the desired texture. The material is then rolled into a ball and allowed to dry for several hours. It may swell to a certain extent and remain soft, but after several hours can be pressed into a firm tablet. Further drying results in a hard tablet ready for packaging.

If the fragrance is used in the form of a liquid, it must be added after the corn syrup or the acid and base combination begins to react. If color is to be applied, an appropriate food coloring dye should be added to the mixture after the corn syrup and immediately before the corn starch. If the dye is added after the corn starch, the result would be that the color will not be distributed evenly and will appear mottled.

The shape of each tablet is dimensioned and configured to be spherical, because this shape provides the maximum surface area, so that whatever the size of the tablet, dissolution time will be maximized. Alternatively, the tablet can be pressed into the shape of a disk. This geometric design is more conducive to packaging and can be transported more easily. Any other desired shape can be utilized simply by using molds for casting. Logos or other markings can then be incorporated into the molds.

All the ingredients listed in Table 2 are non-toxic. Although the deodorizing pellet is not intended to be eaten, swallowed or ingested by humans or animals, each ingredient in the tablet is edible, and thus safe for consumer use. This alleviates some concern about the product being accidently ingested by children and animals. In addition, the small i quantities of each ingredient listed in Table 2 are non-toxic to the environment and biodegradable, so that the composition can be flushed in a toilet fixture.

The deodorizing tablets can be individually packaged, so that one or multiple tablets can be easily carried on one's person or in one's purse. The tablets can be deposited from the packaging directly into the toilet without handling, although if one handles a tablet prior to use, the texture is such that no stickiness or tablet residue will remain on their hands. The size and nature of the tablets makes this composition extremely portable, and provides a sanitary and economical method to eliminate unpleasant odors emanating from the toilet.

One of the unique features of the deodorizing tablet is the portable nature of the composition. This eliminates the need for a permanent deodorizing fixture or device present in the bathroom prior to use of the toilet. This is extremely convenient when the need for a deodorizer arises while the user is away from home.

The primary difference between the deodorizing tablet and similar mounted dispenser products is its portable nature. In a public restroom facility or any other facility in which the user is a guest, a permanent deodorizer may or may not be present. Having a portable convenient deodorizer on one's person ensures that deodorizing capabilities are always at hand. This can prevent uncomfortable or embarrassing situations. The portable nature of the tablet allows one the capability to deodorize restrooms in hotels, offices, buses, planes, or any other location. The single-use deodorizer also provides efficiency. If used on an as-needed basis, the deodorizer is not wasted by continuously emitting a fragrance which may or may not be pleasant in the long term.

This product provides a safe, inexpensive, convenient, and efficient method of deodorizing toilets without having to depend on the property owner. This can aid tremendously in eliminating awkward or embarrassing situations. Finally, this product allows flexibility, in that it can be produced in a variety of colors and/or fragrances.

It is to be understood that the present invention is not limited to the sole embodiments described above, but encompasses any and all embodiments within the scope of the following claims.

We claim:

1. A portable water-soluble deodorizing tablet composition consisting essentially of:
(a) at least one dry water-soluble solid acid;
(b) sodium bicarbonate;
(c) a fragrance; and
(d) at least one binding agent.

2. The tablet composition according to claim 1, wherein said at least one water-soluble acid is selected from the group consisting of citric acid, carbonic acid, acetic acid, ascorbic acid, and benzoic acid.

3. The tablet composition according to claim 1, wherein said at least one water-soluble acid comprises 5% to 15% by weight of said tablet composition.

4. The tablet composition according to claim 1, wherein said sodium bicarbonate comprises from 8% to 18% by weight of said tablet composition.

5. The tablet composition according to claim 1, wherein said fragrance is selected from the group consisting of cinnamaldehyde, vanillin, menthol, menthone, methyl salicylate, isopentyl acetate, ethyl butyrate, octyl acetate, methyl butyrate, pentyl propionate, isobutyl formate, and ethyl formate and mixtures thereof.

6. The tablet composition according to claim 1, wherein said fragrance comprises from 5% to 10% by weight of said tablet composition.

7. The tablet composition according to claim 1, wherein said at least one binding agent is selected from the group consisting of corn syrup and corn starch.

8. The tablet composition according to claim 7, wherein said corn syrup comprises from 20% to 45% by weight of said tablet composition.

9. The tablet composition according to claim 8, wherein said corn syrup comprises from 35% to 45% by weight of said tablet composition.

10. The tablet composition according to claim 1, further including a food coloring dye for adding color to said tablet prior to the addition of the binding liquid.

11. A process for making a deodorizing agent in tablet form comprising the steps of:
(a) combining at least one dry water-soluble acid selected from the group consisting of citric acid, carbonic acid, acetic acid, ascorbic acid, benzoic acid, and mixtures thereof, with dry sodium bicarbonate and dry fragrance in order to form a dry mixture;
(b) adding sufficient corn syrup to the dry mixture to form a cohesive substrate;
(c) adding sufficient corn starch to the cohesive substrate to thicken the mixture and provide a desired texture;
(d) drying the mixture formed in step (c) for several hours; and
(e) compressing the mixture in step (d) into a firm tablet.

12. The process according to claim 11, further comprising the step of adding a food coloring dye after forming the cohesive substrate and before the step of adding the corn starch in order to give said tablet a color for aesthetic value.

13. A process for making a deodorizing agent in tablet form comprising the steps of:
(a) combining dry citric acid and dry sodium bicarbonate in order to form a dry mixture;
(b) adding corn syrup to the dry mixture to form a cohesive substrate;
(c) adding a fragrance in liquid form to the cohesive substrate;
(d) adding corn starch to the cohesive substrate formed in step (c) to thicken the mixture and provide a desired texture;
(e) drying the mixture formed in step (d) for several hours; and
(f) compressing the mixture in step (e) into a firm tablet.

14. The process according to claim 13, further comprising the step of including a food coloring dye for adding color to said tablet prior to the addition of corn starch.
